(19) United States
(12) Patent Application Publication
(10) Pub. No.: US 2013/0110163 A1
(43) Pub. Date: May 2, 2013

(54) ATTACHMENT MECHANISM FOR MATERIAL AND BONE

(71) Applicants: Rodney R. Ballard, Lakeland, TN (US); Chris Johnson, Germantown, TN (US); Newton Metcalf, Memphis, TN (US); Alison Powers, Memphis, TN (US); Joshua Simpson, Collierville, TN (US)

(72) Inventors: Rodney R. Ballard, Lakeland, TN (US); Chris Johnson, Germantown, TN (US); Newton Metcalf, Memphis, TN (US); Alison Powers, Memphis, TN (US); Joshua Simpson, Collierville, TN (US)

(73) Assignee: WARSAW ORTHOPEDIC, INC., Warsaw, IN (US)

(21) Appl. No.: 13/283,886

(22) Filed: Oct. 28, 2011

(51) Int. Cl. A61B 17/04; A61F 2/08

(52) U.S. Cl. 606/232; 623/13.14

(57) ABSTRACT

A method for anchoring a portion of material to bone is disclosed. One such method comprises affixing the portion of material to a neck of a fastener, and driving the fastener into the bone such that the portion of material becomes fixed to the bone.

The present disclosure is related to commonly owned and co-pending U.S. application Ser. No. ______ (having Attorney Docket No. C1370.USU1), which has a filing date that is the same as the present disclosure, and which is hereby incorporated herein by reference in its entirety.

### FIELD OF INVENTION

The present invention is directed to systems or mechanisms for affixing material to bone.

### BACKGROUND

The present disclosure relates to mechanisms for affixing material to bone, and more particularly, systems for affixing at least a portion of material to a vertebral body.

### SUMMARY OF THE INVENTION

A method for anchoring a portion of material to bone is disclosed. One such method comprises affixing the portion of material to a neck of a fastener, and driving the fastener into the bone such that the portion of material becomes fixed to the bone.

Additional aspects and features of the present disclosure will be apparent from the detailed description and claims as set forth below.

### BRIEF DESCRIPTION OF THE DRAWINGS

FIG. 1 is a schematic, isometric view of a system for attaching material to bone;

FIG. 2 is a schematic, isometric view of another system for attaching material to bone;

FIG. 3 is a schematic, isometric view of another system 300 for attaching material to bone;

FIG. 3b is a schematic, isometric view of another system for attaching material to bone; and

FIG. 4 is a schematic, isometric view of another system for attaching material to bone.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

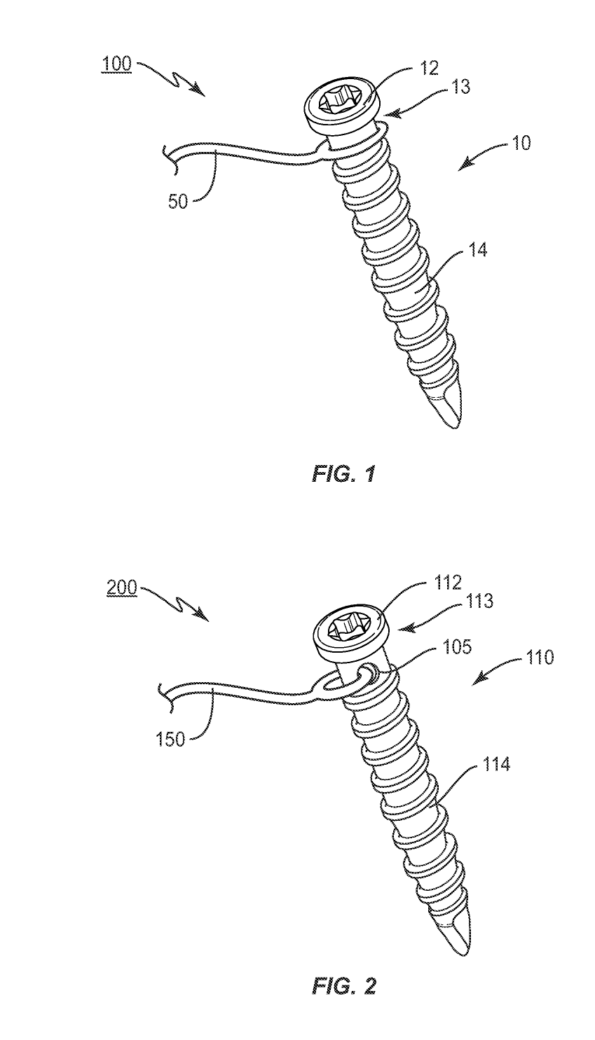

The attachment systems described herein may be used to attach any material to bone, for example, to attach material to vertebral bodies for use in fusionless spinal treatments. FIG. 1 shows a schematic, isometric view of a system 100 for attaching material to bone. Specifically, the system 100 comprises a fastener 10 and a portion of material 50. The fastener 10 comprises a head 12, a neck 13 just below the head 12, and a shank 14. A method for anchoring the portion of material 50 to bone is disclosed. The method comprises affixing the portion of material 50 to the neck 13 of the fastener 10, and then driving the fastener 10 into the bone such that the portion of material 50 becomes fixed to the bone. The portion of material 50 may be affixed to the neck 13 of the fastener 10 by any means know in the art, for example, by gluing, tying a knot or knots, suturing material 50 onto itself, or by effecting a phase change of one or more materials to obtain rigid or solid fusion. The term “affix” is used herein in a relatively broad sense. That is, the word “affix” is intended to mean verbs such as join, secure, and hold. Accordingly, while the word “affix” may encompass the verb fuse, as in fusing together two items in a rigid manner, it is not limited to such a narrow definition.

After the portion of material 50 is affixed to the neck 13 of the fastener 10, the fastener 10 may be driven into bone, for example, a vertebral body. After the fastener 10 is driven into the bone, the fastener 10 will be affixed to the bone and the portion of material 50 also will be affixed to the bone. In this manner, the head 12 of the fastener 10 may help maintain the portion of material 50 affixed to the bone. Further, during the step of driving the fastener, the portion of material 50 may rotate around the fastener 10 such that the portion of material 50 becomes embedded between the fastener 110 and the bone.

FIG. 2 shows a schematic, isometric view of another system 200 for attaching material to bone. Specifically, the system 200 comprises a fastener 110 and a portion of material 150. The fastener 110 comprises a head 112, a neck 113 just below the head 112, and a shank 114. Further, as shown in FIG. 2, the neck 113 comprises a hole 105. A method for anchoring the portion of material 150 to bone is disclosed. The method comprises affixing the portion of material 50 to the neck 13 of the fastener 110 by inserting the portion of material 150 through the hole 105 in the fastener 110, and driving the fastener 110 into the bone such that the portion of material 150 becomes fixed to the bone. As shown in FIG. 2, the portion of material 150 may be affixed to the neck 13 of the fastener 110 by using the hole 105, for example, by tying a knot. Further, securing the portion of material 150 to the fastener 110 may be enhanced by additionally affixing it to the fastener 110 by any means known in the art, for example, by those means described above with reference to system 100.

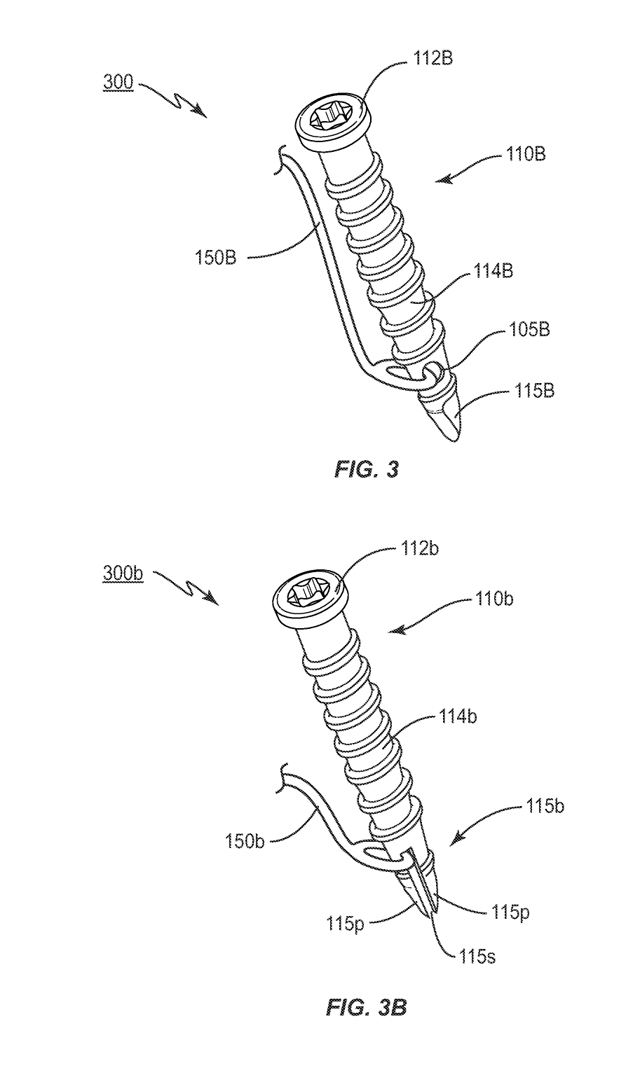

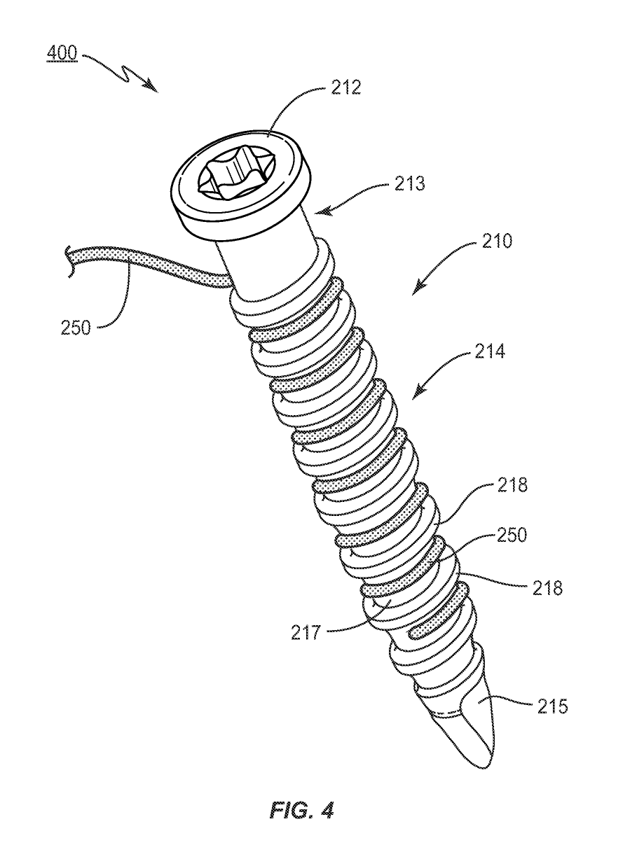

FIG. 3 shows a schematic, isometric view of another system 300 for attaching material to bone. Specifically, the system 300 comprises a fastener 110B and a portion of material 150B. The fastener 110B comprises a head 112B, a shank 114B, and a distal portion 115B. Further, as shown in FIG. 3, the distal portion 115B comprises a hole 105B. A method for anchoring the portion of material 150B to bone is disclosed. The method comprises affixing the portion of material 150B to the distal portion 115B of the fastener 110B by inserting the portion of material 150B through the hole 105B in the fastener 110B, and then driving the fastener 110B into the bone such that the portion of material 150B becomes fixed to the bone. As shown in FIG. 3, the portion of material 150B may be affixed to the distal portion 115B of the fastener 110 by using the hole 105B, for example, by tying a knot. Further, securing the portion of material 150 to the fastener 110 may be enhanced by additionally affixing it to the fastener 110 by any means known in the art, for example, by those means described above with reference to system 100.

Note that with respect to the system 200 of FIG. 2, the hole 105 is in the neck 113 of the fastener 110. With respect to the system 300 of FIG. 3, the hole 105B is in the distal portion 115B of the fastener 110. Note that a hole similar to that of hole 105 or hole 105B may be located in other parts of a fastener, for example, it may be located in the center of the shank. Further, instead of a hole, note that other features may be used to affix a portion of material to a faster. Examples of such features may be a slot or protrusions such as hooks or prongs (similar to that of a tuning fork used in musical arts).

FIG. 3b shows a schematic, isometric view of another system 300b for attaching material to bone. Specifically, the system 300b comprises a fastener 110b and a portion of material 150b. The fastener 110b comprises a head 112b, a shank 114b, and a distal portion 115b. With the system 300b of FIG. 3b, however, the distal portion 115 comprises two prongs 115p and a slot 115s therebetween. Accordingly, with system 300b, the slot 115s and/or one or both prongs 115p may be used to affix a portion of material 150b to the fastener 110b. As shown in FIG. 3b, the fastener 110b is similar to that of a tuning fork used in musical arts.

A system for attaching a portion of material to bone also is disclosed. The system comprises a fastener 110 or 110B, wherein the fastener 110 or 110B comprises a hole 105 or 105B such that the portion of material 150 or 150B may be inserted through the hole 105 or 105B such that driving the fastener 110 or 110B into the bone fixes the portion of material 150 or 150B to the bone.

FIG. 4 shows a schematic, isometric view of another system 400 for attaching material to bone. Specifically, the system 400 comprises a fastener 210 and a portion of material 250. The fastener 250 comprises a head 212, a neck 213, a shank 214, and a distal portion 215. Further, as shown in FIG. 4, the fastener 210 is a screw that has a screw thread for engaging bone. More specifically, the screw thread comprises crests 218 and roots 217 located in between the crests 218. As shown in FIG. 4, the crests have a larger diameter than that of the roots 217. A method for anchoring the portion of material 250 to bone is disclosed. The method comprises placing the portion of material 250 along the roots 217 of the screw 210, between the crests 218, and then driving the screw 210 into the bone such that the portion of material 250 becomes fixed to the bone. Further, securing the portion of material 250 to the fastener 210 may be enhanced by additionally affixing it to the fastener 210 by any means known in the art, for example, by those means described above with reference to system 100. During the step of driving, the portion of material 250 becomes embedded between the screw 210 and the bone.

As shown in FIG. 4, the portion of material 250 is placed along almost the entire length of the shank 214. As another option, the portion of material 250 may be placed along only a portion of the length of the shank 214 or along a majority of the length of the shank 214.

In the attachment systems and methods described herein, the portions of material are non-rigid and flexible. In addition, the portions of material may be a tether or part of a tether. Further, each portion of material may be a ligament, i.e., synthetic ligament or natural ligament. In addition, each portion of material may be a graft such as an autograft, allograft or xenograft. Also, within the spirit of the invention, any of the fasteners described herein may be screws or other fasteners such as dowels, and such dowels may be made of synthetic material or made of natural materials such as bone, or a combination comprised of both a synthetic material and a biologic material, or a biologic material that may be considered more than minimally processed.

Further, the portions of material may be any one or combination of a cloth, metal, solid polymer, fabric, mesh, or other biocompatible material. Some polymer materials may include but not be limited to, any one or combination of polyethylene, polyester, polyvinyl, polyvinyl alcohol, polyacrylonitrile, polyamide, polytetrafluoroethylene, polyparaphenylene and terephthalamide. In addition, the portions of material may be made of a suture wire of polyester or polyethylene. Further, the portions of material may be elastic, woven, knitted, braided or flexible. Some woven, knitted or braided materials may, for example, include nylon, Dacron®, and/or woven fibers or filaments of polyester, polyethelene, polypropylene, polyetheretherketone (“PEEK”), polytetrafluoroethylene (“PTFE”), and/or woven PEEK. Some elastic materials may, for example, include latex, rubber, silicone, polyurethane, silicone-polyurethane copolymers, and/or polyolefin rubbers. Other suitable materials may, for example, include Gore-Tex®, Kevlar®, Spectra, polyether, polycarbonate urethane, shape memory material with pseudo elastic or superelastic characteristics, metals, metal alloys, and polymers, braided polymers, synthetic resorbable materials such as polyactide, polygycolide, polyorthoester, calcium phosphate, and/or glass, nonresorbable polyethylene, cellulose, materials that are potentially absorbable, and/or materials that are used in making synthetic ligaments. Further, suitable materials may be biodegradable or non-biodegradable. Similarly, suitable materials may be resorbable or and/or non-resorbable. In addition to woven, braided, or knitted structures, the portions of material also may be composed of non-woven structures such as non-woven mesh or chained structures.

All adjustments and alternatives described above are intended to be included within the scope of the invention, as defined exclusively in the following claims. Those skilled in the art also should realize that such modifications and equivalent constructions or methods do not depart from the spirit and scope of the present disclosure, and that they may make various changes, substitutions, and alterations herein without departing from the spirit and scope of the present disclosure. For example, although the attachment systems above are described as being configured to affix a portion of material to a vertebral body, they also may affix a portion of material to any bone. Similarly, although the attachment systems are described as being configured to affix to a pedicle of a vertebral body, they also may affix to other surfaces of a vertebral body such as an anterior, antero-lateral, or lateral face of a vertebral body or any plurality or combination of such surfaces.

Furthermore, as used herein, the terms components and elements may be interchanged. It is understood that all spatial references, such as “superior,” “inferior,” “anterior,” “posterior,” “above,” “lower,” “outside,” “inside,” “higher,” “lower,” “outer,” “inner,” “extended,” “reduced,” “shorter,” “longer,” and “perimeter” are for illustrative purposes and can be varied within the scope of the disclosure.

1. A method for anchoring a portion of material to bone, the method comprising:
affixing the portion of material to a neck of a fastener; and
driving the fastener into the bone such that the portion of material becomes fixed to the bone.

2. The method of claim 1, wherein during the step of driving, the portion of material rotates around the fastener such that the portion of material becomes embedded between the fastener and the bone.

3. The method of claim 1 further comprising a step of inserting the portion of material through a hole in the fastener.

4. The method of claim 3, wherein the step of affixing is performed before the step of driving.

5. The method of claim 4, wherein the step of inserting is performed before the step of affixing.

6. The method of claim 3, wherein the hole is in the neck of the fastener.

7. The method of claim 3, wherein the hole is in the distal portion of the fastener.

8. The method of claim 1, wherein the fastener is a screw.

9. The method of claim 1, wherein the portion of material is flexible.

10. The method of claim 9, wherein the portion of material is part of a tether.

11. The method of claim 1, wherein the portion of material is a ligament.

12. The method of claim 11, wherein the portion of material is a synthetic ligament.

13. The method of claim 1, wherein the portion of material is an allograft.

14. The method of claim 5, wherein the portion of material is a ligament.

15. A method for anchoring a portion of material to bone, the method comprising:
placing the portion of material along a root of a screw, wherein the root is the part of the screw between the crests of the screw; and
driving the screw into the bone such that the portion of material becomes fixed to the bone.

16. The method of claim 15, wherein the screw has a shank and the shank has a length, and during the step of placing, the portion of material is placed along a majority of the length of the shank.

17. The method of claim 15, wherein during the step of driving, the portion of material becomes embedded between the screw and the bone.

18. The method of claim 15, wherein the portion of material is flexible.

19. A system for attaching a portion of material to bone, the system comprising:
a fastener, wherein the fastener comprises a hole such that the portion of material may be inserted through the hole such that driving the fastener into the bone fixes the portion of material to the bone.

20. The system of claim 19, wherein the hole is located in a distal portion of the fastener.
